(19) United States
(12) Patent Application Publication
(10) Pub. No.: US 2013/0096465 A1
(43) Pub. Date: Apr. 18, 2013

(54) Method and Apparatus for Measuring Volume of Subcutaneous Tumors

(71) Applicants: Allan H. Conney, Princeton, NJ (US); Xi Zheng, Edison, NJ (US)

(72) Inventors: Allan H. Conney, Princeton, NJ (US); Xi Zheng, Edison, NJ (US)

(21) Appl. No.: 13/624,346

(22) Filed: Sep. 21, 2012

Related U.S. Application Data
(60) Provisional application No. 61/537,289, filed on Sep. 21, 2011.

(51) Int. Cl. A61B 5/107

(52) U.S. Cl. CPC A61B 5/1075
USPC 600/587

(57) ABSTRACT

A method and apparatus for measuring tumors such as subcutaneous tumors is provided. A device having a flexible membrane is positioned over the tumor and displaced toward the tumor so that the tumor deforms the flexible membrane. Deforming the membrane changes the pressure or volume within a fluid-filled cavity. A gauge measures the change in volume or pressure and the gauge reading correlates with the volume of the tumor.

### PRIORITY CLAIM

This application claims priority to U.S. Provisional Patent Application No. 61/537,289, filed Sep. 21, 2011. The entire disclosure of the foregoing application is hereby incorporated by reference.

### FIELD OF THE INVENTION

This present invention relates to the field of measuring tumors. More specifically, the present invention relates to the field of measuring the volume of a tumor. In particular, the present invention relates to volumetric measurement of subcutaneous tumors.

### BACKGROUND OF THE INVENTION

Measuring tumor size is a common aspect of preclinical studies when assessing responses to cancer treatment. Typically, it is desirable to use a non-invasive technique to obtain a series of measurements over the course of the study. The current standard technique for determining volume of subcutaneously tumors in vivo is by external caliper. The calipers are used to measure the length and width of the tumor and the tumor volume is estimated by use of the modified ellipsoid formula ½(Length×Width2). This method uses two dimensional measurements (length and width) to estimate a three dimension outcome (volume). Because the height of the tumor is not measured, calculation of tumor volume using this formula is only approximate. The height of a subcutaneous tumor is difficult to measure by a standard caliper. Moreover, measurements of length and width using a caliper are often affected by errors due to the irregularity of tumor shape. Accordingly, there exists a need for an accurate, efficient, non-invasive technique for measuring subcutaneous tumors.

### SUMMARY OF THE INVENTION

In light of the shortcomings of the prior art, the present invention provides an apparatus for accurately and efficiently measuring the volume of subcutaneous tumors. According to one aspect, the present invention includes a fluid-filled housing. A flexible membrane forms a wall of the fluid filled housing. A measuring element measures the change in volume or pressure within the housing when the flexible membrane is stretched over a subcutaneous tumor.

According to another aspect, the present invention provides a method of measuring the volume of subcutaneous tumors using a fluid-filled device having a flexible wall. According to the method, a flexible wall of the fluid-filled device is placed onto a subcutaneous tumor. The device is displaced toward the subcutaneous tumor so that the tumor deforms the flexible wall. The change in volume or pressure within the fluid-filled device in response to the deformation of the flexible wall is then measured.

### BRIEF DESCRIPTION OF THE DRAWINGS

The foregoing summary and the following detailed description of the preferred embodiments of the present invention will be best understood when read in conjunction with the appended drawings, in which:

FIG. 1 is a side sectional view of a device for measuring subcutaneous tumors;

FIG. 2 is an exploded side view of the device illustrated in FIG. 1;

FIG. 3 is a side sectional view of the device illustrated in FIG. 1, shown overlaying a subcutaneous tumor;

FIG. 4 is a side sectional view of an alternate device for measuring subcutaneous tumors;

FIG. 5 is a side sectional view of the device illustrated in FIG. 4, shown overlaying a subcutaneous tumor.

FIG. 6 is a side view of an alternate device for measuring subcutaneous tumors; and

FIG. 7 is a device for calibrating the devices illustrated in FIGS. 1-6.

### DETAILED DESCRIPTION OF THE INVENTION

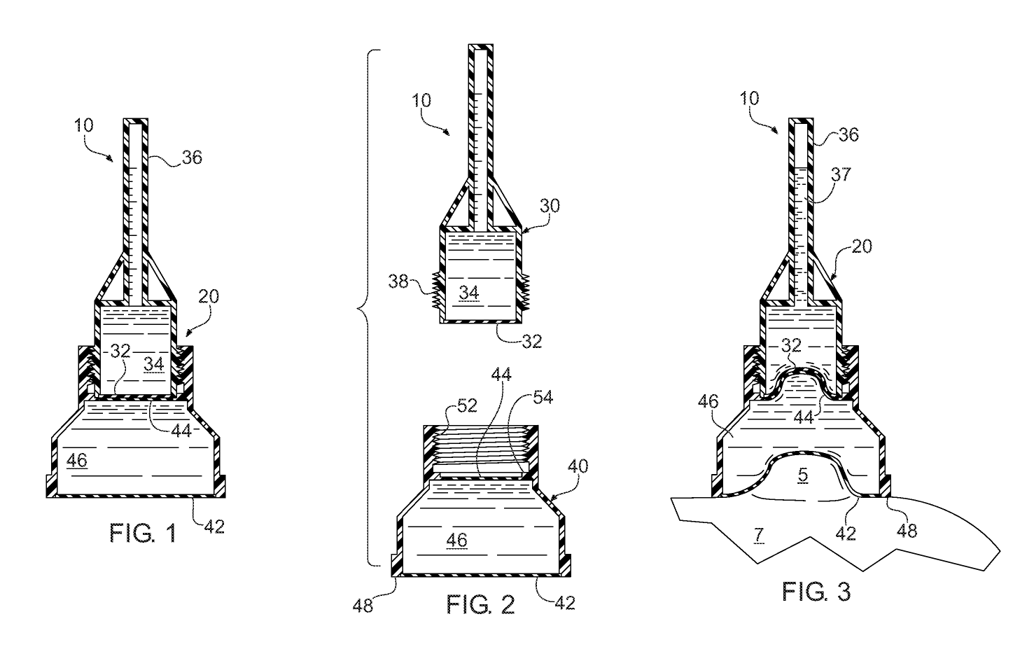

Referring now to the figures in general, wherein like elements are numbered alike throughout, an apparatus for measuring subcutaneous tumors is designated generally 10. As shown in FIGS. 1-3, the measuring device 10 can be placed over a tumor to measure the volume of the tumor. The tumor deforms a flexible wall, such as a membrane 32, thereby displacing fluid within the housing so that fluid from the housing flows into a gauge 36. The fluid level in the gauge 36 is proportional to the volume of the tumor.

The device 10 comprises a fluid-filled housing 20. The housing may be formed as a unitary or single-piece housing. However, in the present instance, the housing 20 is a two-piece structure formed by an upper housing 30 and a lower housing 40 that are releasably connectable as discussed further below. By using a two-piece construction, the upper housing 30 and gauge 36 can be used with a variety of differently configured lower housings 40 depending on the size or configuration of the tumor to be measured.

Referring to FIG. 2, the upper housing 30 comprises a generally cylindrical hollow housing having a fluid-filled cavity 34. The fluid in the cavity may be either a gas or an incompressible liquid, such as water. The upper housing 30 comprises a connector 38 for connecting the upper housing with the lower housing 40. The connector 38 may be any of a variety of releasable connectors, such as interlocking grooves, quick connectors or other mechanical connectors. In the present instance, the connector 38 comprises external screw threads.

The upper housing further includes a resiliently flexible lower wall 32 sealing the fluid within the cavity 34. The flexible wall is taut and may be formed of a variety of soft readily deformable materials, such as rubber or plastic. In the present instance, the flexible wall 32 is a rubber or plastic membrane. A gauge 36 attached to the upper housing 30 provides an indicator of change in pressure or volume within the cavity 34. In the present instance, the gauge comprises an elongated narrow tube 36 in fluid communication with the fluid cavity 34. As the membrane 32 is deformed into the fluid cavity 34, fluid from the fluid cavity is forced into the gauge 36. The volume of fluid flowing into the gauge is denoted 37 in FIG. 3, and it correlates with the volume of the tumor being measured, as discussed further below. The gauge 36 includes markings or indicators, such as a graduated cylinder, to indicate the volume of fluid based on the level of the fluid in the gauge. The gauge may be a sealed container or alternatively, the gauge may include a gas-permeable filter or seal that is substantially impermeable to liquid. In this way, gas such as air can evacuate the upper housing 30, but liquid will remain sealed in the upper housing.

Referring again to FIGS. 1 and 2, the lower housing 40 comprises a hollow housing having a fluid-filled cavity 46. The fluid in the cavity may be either a gas or an incompressible liquid, such as water. The lower housing comprises a connector 52 cooperable with the connector 38 of the upper housing 30 to releasably connect the lower housing 40 with the upper housing 30. In the present instance, the connector 52 comprises internal screw threads that cooperate with the external screw threads 38 of the upper housing 30.

The lower housing includes a resiliently flexible upper wall 44 that seals the upper end of the lower housing 40. The flexible wall 44 is taut and may be formed of a variety of soft readily deformable materials, such as rubber or plastic. In the present instance, the flexible wall 44 is a rubber or plastic membrane. The lower housing may include an annular flange 54 extending radially inwardly from the inner wall of the lower housing, adjacent the internal threads 52. The upper membrane 44 may connect with the annular flange 54 and stretch inwardly extending across the open upper end of the lower housing. Specifically, the upper membrane 44 stretches across the width of the upper end of the lower housing and is positioned so that when the lower housing 40 is connected with the upper housing 30, the upper membrane 44 is adjacent the membrane 32 of the upper housing, as shown in FIG. 1. In this way, deformation of the upper membrane 44 also deforms the membrane 32 of the upper housing.

The lower housing 40 may be formed in a variety of shapes and sizes. In the present instance the lower housing is generally cylindrical, having a frusto-conical upper portion that tapers down to a diameter mating with the upper housing 30. Depending on the shape and size of the tumor to be measured, the lower housing 40 may have a larger diameter or a smaller diameter to accommodate the tumor.

The lower end of the lower housing 40 forms a rim 48 that circumscribes the bottom of the lower housing. A lower membrane 42 extends across the bottom surface of the lower housing, sealing the lower end of the lower housing 40. In the present instance, the lower membrane 42 is taut and extends from the rim 48 of the lower housing.

Configured as described above, the device 10 operates as follows. The operator selects a lower housing 40 that is sized so that the rim 48 of the lower housing circumscribes the width of the tumor 5 to be measured. The lower housing 40 is then connected with the upper housing 30, such as by threadedly connecting the lower housing with the upper housing. When attached, the membrane 32 of the upper housing 30 overlies the upper membrane 44 of the lower housing 40. In the present instance, the membrane 32 of the upper housing is in contact with or minimally separated from the upper membrane 44 (e.g. approximately a millimeter or less gap between the membranes). The operator then places the device 10 onto the tumor 5 by positioning the lower membrane 42 on top of the tumor so that the rim circumscribes the tumor. The operator then pushes the device toward the tumor until the rim 48 of the device engages the epidermis 7.

As the operator displaces the device 10 toward the tumor 7, the tumor deforms the lower membrane 42 inwardly into the fluid cavity 46 as shown in FIG. 3. The side walls are sufficiently rigid relative to the upper and lower membranes 42, 44. Therefore, when the lower membrane 42 is deformed inwardly by the tumor 7, the fluid in the fluid cavity 46 deforms the upper membrane 44 outwardly toward the upper fluid cavity 34. In turn, the upper membrane 44 deforms the membrane 32 at the lower edge of the upper housing 30, thereby displacing fluid from the upper fluid cavity 34. The fluid displaced from the upper fluid cavity flows into the gauge 36. The operator can then read the fluid level 37 in the gauge 36 while the device 10 is positioned over the tumor as shown in FIG. 3. The fluid level will correlate with the volume of the tumor 7.

In the foregoing description, the device has been described as having a two-part housing so that configuration of the lower portion of the device can be changed depending on the size and shape of the tumor to be measured. Alternatively, the housing 20 may be formed as a unitary element. Doing so eliminates the need for the connectors 38, 52 and the internal membranes 32, 44. The operation of the device will be substantially the same as described above. Specifically, the operator places the device over the tumor so that the membrane at the bottom of the device overlies the tumor. The device is then displaced toward the tumor so that the tumor deforms the membrane at the bottom of the device. As the tumor deforms the membrane inwardly into the fluid cavity, fluid is forced from the fluid cavity into the gauge and the operator can measure the fluid level in the gauge, which corresponds to the volume of the tumor.

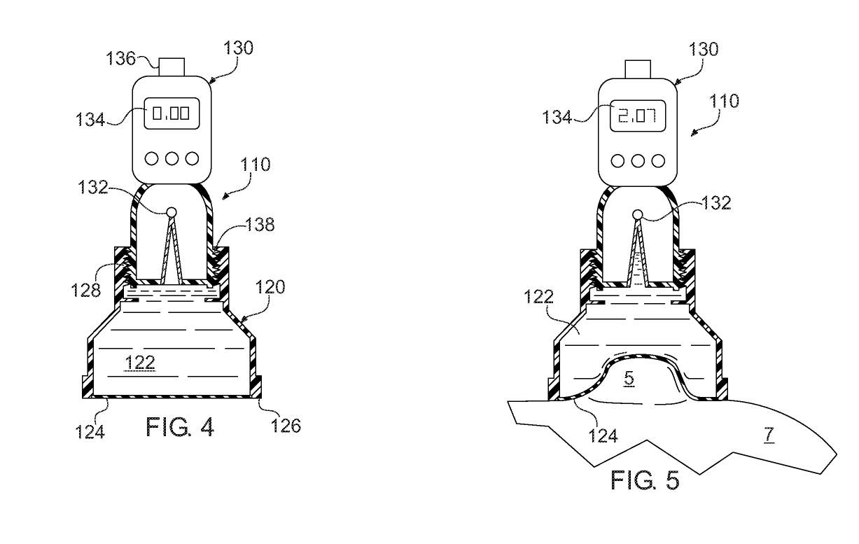

Referring now to FIGS. 4-5, an alternative embodiment is illustrated. The device 110 is operable to measure the volume of a subcutaneous tumor 5. The device measures the volume of the tumor based on a change in the pressure in a fluid-filled cavity.

The device 110 includes a housing 120, which is generally cylindrical and has a hollow interior forming a fluid cavity 122. The side walls of the housing are generally rigid and may be formed of any of a variety of rigid or semi-rigid materials, such as plastic or metal. The lower end of the housing forms a rim 126 similar to the rim 48 of the device 10 described above. Additionally, the lower end of the housing is sealed by a taut flexible wall 124 that is formed substantially similar to the membrane 42 of the device 10 described above.

A pressure gauge 130 is positioned in the upper end of the housing 120 so that the pressure gage is in fluid communication with the fluid cavity 122 in the housing. The upper end of the housing is closed to form a fluid tight seal between the fluid cavity 122 and the atmosphere external to the housing. In the present instance, the upper end of the housing comprises a connector 128, such as internal threads, that cooperate with a connector 138 on the pressure gauge 130, such as external threads. In this way, the threaded connection between the housing 120 and the gauge forms a fluid-tight seal to prevent fluid from migrating out of the fluid cavity.

The pressure gauge 130 may be any of a variety of gauges. In the present instance, the pressure gauge is a digital gauge having a readily viewable display 134, which displays the pressure reading. The gauge may also include a data port 136 so that data from the pressure gauge can be output to a remote device, such as a computer. For instance, the pressure gauge may include a data port such as a USB connector for connecting the gauge to a remote device for processing or recording the data regarding the pressure readings. The pressure gauge may include a processor that is programmable to correlate the change in pressure to a corresponding change in volume for the fluid cavity, which in turn would correspond with the tumor volume. Alternatively, the data from the pressure gauge can be exported to a remote device, such as a computer, and the computer can process the data to correlate the pressure changes to calculate the tumor volume.

Configured as described above, the device is operable as follows. The operator may select a housing 120 appropriately sized depending on the size and configuration of the subcutaneous tumor to be measured. Specifically, the housing may be selected so that the diameter of the rim 126 of the housing completely circumscribes the width of the tumor 7. A pressure gauge 130 is then connected to the housing, such as by threadedly connecting the pressure gauge to the housing. To measure a tumor, the device 110 is positioned over a tumor 7 so that the membrane 124 overlies the surface of the tumor. The operator then displaces the device 110 toward the tumor until the rim 126 engages the epidermis 7 adjacent the tumor as shown in FIG. 5. As the device is displaced toward the tumor 7, the tumor deforms the membrane 124 inwardly into the fluid cavity 122. Since the side walls of the housing 120 are substantially rigid, deforming the membrane inwardly reduces the volume in the fluid cavity, thereby increasing the pressure within the cavity. The sensor 132 of the pressure gauge 130 is in fluid communication with the fluid cavity 122 so that the sensor detects the increase in pressure as the membrane is deformed inwardly by the tumor.

The sensor displays the change in pressure as the device 110 is displaced over the tumor to deform the membrane. In this way, the operator can record the pressure created within the fluid cavity when the device is placed over the tumor until the rim engages the epidermis. The operator can then correlate the pressure with the volume of the tumor via either a calculation or a reference chart that correlates pressure differential with tumor volume. Alternatively, the pressure gauge may be programmed so that the gauge includes a processor operable to process the pressure readings and convert the pressure reading to the corresponding volume. Further still, as described above, the gauge 130 may include a data port 136 so that the pressure data and/or the volume data can be exported to a remote device and stored or further processed on the remote device.

As described above, the pressure gauge may be releasably connected with the housing so that a variety of housing sizes and configurations can be utilized. Alternatively, the pressure gauge may be substantially permanently connected with the housing so that a standardized housing size is used for each measurement.

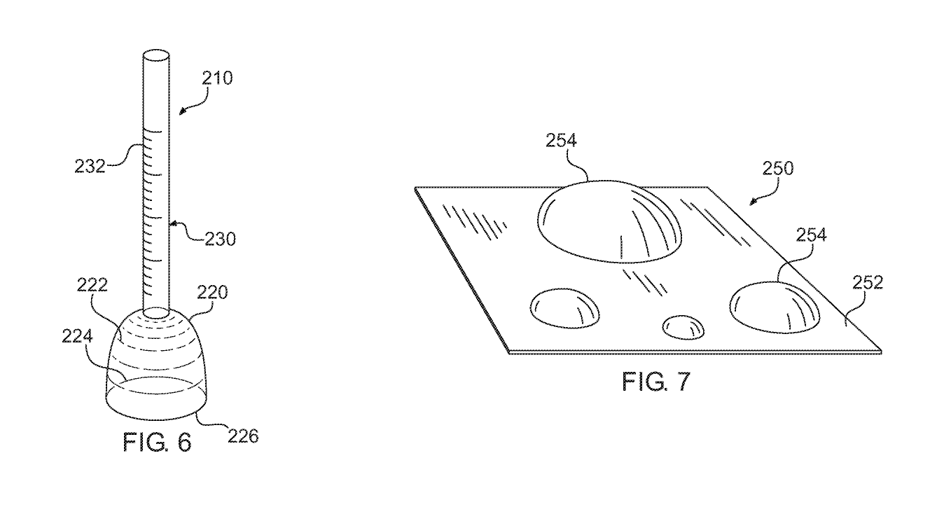

Referring now to FIG. 6 an alternative embodiment of a tumor measuring device is designated 210. The device 210 comprises a hollow housing 220 having a sealed fluid cavity 222. The upper and side walls of the housing are formed of generally rigid materials, such as plastic. The lower edge of the housing side wall forms a rim 226. The lower wall of the housing 224 is a resiliently flexible wall that is readily deformable by soft tissue, such as a subcutaneous tumor. For instance, the lower wall 224 may be formed of rubber, soft plastic or silica. The device 210 includes a gauge 230 in fluid communication with the fluid cavity 222. In the present instance, the gauge is a fluid gauge measuring the level of fluid displaced out of the fluid cavity and into the gauge. Specifically, the gauge is an elongated cylindrical tube having a diameter smaller than the fluid cavity. The tube includes graduated markings 232 designating the measure the volume of fluid in the fluid gauge.

To use the device 210, the operator positions the device over a subcutaneous tumor so that the rim 226 of the housing circumscribes the tumor. The operator displaces the device toward the epidermis adjacent the tumor. As the device is displaced toward the epidermis, the tumor deforms the flexible wall 224, deforming the wall inwardly into the fluid cavity 222. The deformed wall 224 reduces the volume within the fluid cavity, thereby displacing fluid from the fluid cavity 222 into the gauge 230. The volume of fluid displaced into the gauge can be read using the markings 232 on the gauge, and the volume of fluid in the gauge correlates with the volume of the subcutaneous tumor.

Although the device 210 is illustrated as using a fluid gauge, it should be understood that a variety of gauges can be used that monitor changes in volume or changes in pressure in the fluid cavity. For instance, the gauge 230 may comprise a pressure gauge, such as a typical tire pressure gauge, which displaces an indicator in response to a change in pressure. Similarly, the gauge can be a pressure gauge having a sensor and a digital or analog display for displaying the pressure change in response to a tumor deforming the flexible wall 224. As discussed above, if a digital gauge is used, the gauge may include an outlet port for communicating the data from the gauge with a remote device, such as a computer, to store and/or process the data.

Referring now to FIG. 7 a calibration device 250 is illustrated. The device 250 includes a generally planar surface 252 and a plurality of protuberances 254 projecting from the planar surface. The protuberances are variously sized, and each protuberance has a known volume. In this way, the device 250 can be used to calibrate the tumor measuring devices described above.

To calibrate a device, the device is positioned over one of the protuberances 254 and displaced toward the protuberance so that the protuberance deforms the flexible lower wall of the device, thereby causing the gauge to measure the change in pressure or volume of the device. For example, referring to FIGS. 6-7, the device 210 is placed onto a protuberance 254 and pressed against the protuberance so that the protuberance deforms the flexible wall 224 inwardly into the fluid cavity 222. The device 210 is displaced toward the protuberance until the rim 226 engages the planar surface 252 of the calibration device 250. At this point, the fluid level in the gauge 230 should correspond with the known volume of the protuberance being measured. If there is any variation between the volume as measured by the gauge 230 and the expected volume based on the known volume of the protuberance, the gauge can be calibrated to match the correct measurement.

It will be recognized by those skilled in the art that changes or modifications may be made to the above-described embodiments without departing from the broad inventive concepts of the invention. It should therefore be understood that this invention is not limited to the particular embodiments described herein, but is intended to include all changes and modifications that are within the scope and spirit of the invention as set forth in the claims.

What is claimed is:

1. A method for measuring the volume of subcutaneous tumors using a fluid-filled device having a flexible wall, comprising the steps of:
placing the flexible wall of the fluid filled device onto a subcutaneous tumor;
displacing the fluid filled device toward the subcutaneous tumor so that the tumor deforms the flexible wall;
measuring the change in volume or pressure of the fluid within a cavity of the fluid-filled device.

2. The method of claim 1 wherein the fluid-filled device comprises a rim and the step of displacing comprises displacing the fluid-filled device toward the subcutaneous tumor until the rim engages the epidermis adjacent the tumor.

3. The method of claim 1 wherein the step of measuring comprises measuring the change in volume in the cavity.

4. The method of claim 1 wherein the step of displacing the fluid-filled device toward the subcutaneous tumor deforms the flexible wall, thereby displacing fluid from the cavity into a gauge.

5. The method of claim 4 wherein the step of measuring comprises measuring the amount of fluid displaced into the gauge.

6. The method of claim 1 wherein the step of displacing the fluid-filled device toward the subcutaneous tumor deforms the flexible wall thereby increasing the pressure within the cavity.

7. The method of claim 6 wherein the step of measuring comprises measuring the increase in pressure within the cavity in response to deforming the flexible wall.

8. A device for measuring the volume of a subcutaneous tumor, comprising:
a housing comprising a fluid cavity;
a flexible membrane forming a wall of the housing;
a gauge in fluid communication with the fluid cavity wherein the volume of a subcutaneous tumor can be measured by placing the housing over the tumor so that the tumor deforms the flexible membrane thereby changing the pressure or volume of fluid in the fluid cavity and wherein the gauge measures the change in pressure or volume in the fluid cavity

9. The device of claim 8 wherein the fluid cavity is filled with liquid and deforming the membrane displaces fluid from the cavity into the gauge.

10. The device of claim 8 wherein the gauge comprises a pressure sensor for detecting pressure change in the fluid cavity in response to deforming the membrane.

11. The device of claim 8 wherein the housing comprises an upper housing and a lower housing, and wherein the fluid cavity is formed in the upper cavity and a second fluid cavity is formed in the lower cavity.

12. The device of claim 11 wherein the membrane forms a wall of the lower housing and wherein the upper housing comprises a second membrane forming a wall of the fluid cavity.

13. The device of claim 11 wherein the upper housing and lower housing are releasably connectable.

14. The device of claim 8 wherein the housing comprises a generally rigid rim and the membrane extends from the rim.
